(19) United States
(12) Patent Application Publication
(10) Pub. No.: US 2015/0099989 A1
(43) Pub. Date: Apr. 9, 2015

(54) BLOOD FLOW RATE MEASUREMENT DEVICE

(71) Applicant: TERUMO KABUSHIKI KAISHA, Shibuya-ku (JP)

(72) Inventor: Risato KOBAYASHI, Hadano-city (JP)

(21) Appl. No.: 14/509,600

(22) Filed: Oct. 8, 2014

(30) Foreign Application Priority Data
Oct. 8, 2013 (JP) 2013-211153

(51) Int. Cl. A61B 5/026; A61B 18/14

(52) U.S. Cl. CPC A61B 5/026; A61B 18/1492; A61B 2018/00214
USPC 600/505; 606/40; 606/49

(57) ABSTRACT

A blood flow rate acquisition device which does not require an injection operation for injecting a cooling saline into a blood vessel and can acquire a blood flow rate inside the blood vessel includes an elongated member that is insertable into a blood vessel, a flow velocity measuring member that is disposed in a distal end of a distal end portion of the elongated member in order to measure a flow velocity of blood flow, a cross-sectional area measuring member that is disposed on a side surface in the distal end portion of the elongated member in order to measure a cross-sectional area of a lumen of the blood vessel, and an acquisition device that acquires the blood flow rate by using detection values detected by the flow velocity measuring member and the cross-sectional area measuring member.

### CROSS-REFERENCES TO RELATED APPLICATIONS

This application claims priority to Japanese Application No. 2013-211153 filed on Oct. 8, 2013, the entire content of which is incorporated herein by reference.

### TECHNICAL FIELD

The present disclosure relates to a blood flow rate measurement device, and particularly relates to a blood flow rate measurement device for use during a medical procedure.

### BACKGROUND DISCUSSION

In the field of medicine, various types of treatments and examinations are performed using an elongated hollow tube called a catheter. Examples include a method of directly administering a drug to an affected site through a catheter, a method of pressing, widening and opening a stenosis in a body lumen by using a catheter in which a dilating balloon is attached to a distal end thereof, and a method of scraping and opening the affected site using a catheter in which a cutter is attached to a distal end portion thereof.

In medical treatments which use such catheters to carry out acardiac clinical treatment or surgery, a blood flow velocity or a blood flow rate is often measured in each region inside the heart in order to monitor conditions of the patient.

Japanese Patent No. 3116032 discloses a guidewire-type blood flow meter in which a stainless steel-made guidewire whose outer diameter is 0.3 mm to 1.1 mm and which has a lumen is a helical and tubular coil formed by densely winding platinum-made fine wires in which a section having a length of 10 mm to 30 mm including a rounded portion in a distal end thereof is X-ray opaque. The guidewire-type blood flow meter has a configuration in which a fine wire connecting an external device and a temperature sensing member disposed in an opening of the guidewire is internally provided in the lumen of the guidewire. The guidewire-type blood flow meter causes a temperature sensing unit in the opening to sense a temperature of blood flow which has been thermally diluted by a cooling saline injected through a flexible capillary, and measures the blood flow velocity and the blood flow rate by using a temperature change curve diagram of the blood flow which has been autographically recorded on the external device.

### SUMMARY

However, because it is necessary to inject the cooling saline into the blood vessel and to perform an injection operation for thermally diluting the blood flow in the guidewire-type blood flow meter discussed above, it is difficult to immediately measure the blood flow rate inside the blood vessel.

The present application disclose a blood flow rate measurement device which does not require an injection operation for injecting a cooling saline into a blood vessel in order to acquire a blood flow rate inside the blood vessel.

A blood flow rate measurement device according to an embodiment includes an elongated member that is insertable into a blood vessel, a flow velocity measuring member that is disposed in a distal end of a distal end portion of the elongated member in order to measure a flow velocity of blood flow, a cross-sectional area measuring member that is disposed on a side surface in the distal end portion of the elongated member in order to measure a cross-sectional area of a lumen of the blood vessel, and an acquisition device that acquires the blood flow rate by using detection values detected by the flow velocity measuring member and the cross-sectional area measuring member.

According to an embodiment, the flow velocity measuring member includes a flow velocity measuring transducer.

According to an embodiment, the flow velocity measuring transducer is attached to the distal end of the elongated member so as to radiate ultrasonic waves in a direction parallel to a blood flow direction inside the blood vessel.

According to an embodiment, the cross-sectional area measuring member includes a pair of annular electrodes surrounding the side surface of the elongated member in a circumferential direction, and the pair of annular electrodes are arranged to be apart from each other in an extending direction of the elongated member.

According to an embodiment, the cross-sectional area measuring member includes a cross-sectional area measuring transducer which generates ultrasonic waves in a direction substantially orthogonal to an extending direction of the elongated member, and the cross-sectional area measuring transducer is disposed at multiple locations in the circumferential direction of the side surface of the elongated member.

According to an embodiment, the blood flow rate measuring device may further include a stabilizing member that stabilizes a position of the distal end portion of the elongated member inside the blood vessel. In the embodiment, the stabilizing member is an expansion member which expands at a predetermined position inside the blood vessel and comes into contact with an inner wall of the blood vessel.

According to an embodiment, the cross-sectional area measuring member includes a cross-sectional area measuring transducer which generates ultrasonic waves in a direction substantially orthogonal to an extending direction of the elongated member, and the elongated member is rotatable, thereby enabling the ultrasonic waves to be generated toward a whole region of an inner wall of the blood vessel in the circumferential direction.

According to an embodiment, the blood flow rate measurement device may further include a stabilizing member that stabilizes a position of the distal end portion of the elongated member inside the blood vessel. In the embodiment, the stabilizing member has a substantially truncated cone shape in which a hollow portion penetrating a top surface and a bottom surface is divided, the elongated member extends penetrating the hollow portion, and the stabilizing member is attached to the side surface of the elongated member so that an outer diameter of a cross section orthogonal to the extending direction gradually increases toward the distal side in the extending direction of the elongated member.

According to embodiments of the blood flow rate measurement device, it is possible to realize a medical treatment method including a process of guiding the flow velocity measuring member for measuring the blood flow velocity inside the blood vessel and the cross-sectional area measuring member for measuring the cross-sectional area of the blood vessel to a predetermined position inside the blood vessel, a process of guiding a neural activity stop device which stops a neural activity of a nerve located in the vicinity of an outer wall of the blood vessel to the vicinity of the predetermined position inside the blood vessel, a process of starting an operation for causing the neural activity stop device to stop the neural activity and monitoring the blood flow rate acquired by using the detection values detected by the flow velocity measuring member and the cross-sectional area measuring member during the operation, and a process of determining whether the neural activity stop device has completed the operation according to the monitored blood flow rate.

An embodiment of a medical treatment method includes: inserting an elongated member of a blood flow measurement device into a blood vessel; measuring, with the blood flow measurement device, a flow rate of blood within the blood vessel; performing a procedure on a portion of the blood vessel; measuring, with the blood flow measurement device, the flow rate of blood within the blood vessel after performing the procedure; and comparing the measured flow rate of blood within the blood vessel before performing the procedure with the measured flow rate of blood within the blood vessel after performing the procedure.

In an embodiment of the medical treatment method, the elongated member of the blood flow measurement device remains inserted in the blood vessel while the procedure is performed, and the method further comprises measuring the flow rate of blood within the blood vessel while the procedure is performed.

In an embodiment of the medical treatment method, the elongated member of the blood flow measurement device remains inserted in the blood vessel while the procedure is performed, and the method further comprises measuring the flow rate of blood within the blood vessel while the procedure is performed.

In an embodiment of the medical treatment method, the procedure comprises denervation of a nerve of an outer wall of the blood vessel, and the denervation of the nerve is performed using a cauterizing electrode disposed on an outer surface of an expansion body.

In an embodiment of the medical treatment method, the acquisition device calculates the blood flow rate by using the detection values detected by the flow velocity measuring member and the cross-sectional area measuring member.

In an embodiment of the medical treatment method, the neural activity stop device completes the operation when the monitored blood flow rate reaches a predetermined amount or more.

In an embodiment of the medical treatment method, a blood pressure is monitored in addition to the monitoring of the blood flow rate.

In an embodiment of the medical treatment method, the flow velocity measuring member is provided in the distal end of the distal end portion, and the distal end portion of the elongated member including the cross-sectional area measuring member on the side surface of the distal end portion is guided to the predetermined position inside the blood vessel.

In an embodiment of the medical treatment method, a process of extracorporeally removing the neural activity stop device and the elongated member from the inside of the blood vessel, after the neural activity stop device completes the operation, is included.

According to embodiments of the blood flow rate measurement device, it is not necessary to perform an injection operation for injecting a cooling saline into a blood vessel, and it is possible to acquire a blood flow rate inside the blood vessel.

### BRIEF DESCRIPTION OF THE DRAWINGS

FIG. 1 is a view illustrating a blood flow rate measurement device 1 according to a first embodiment.

FIG. 2 is a view illustrating a blood flow rate measurement device 31 according to a second embodiment.

FIG. 3 is a view illustrating a blood flow rate measurement device 41 according to a third embodiment.

FIG. 4 is a view illustrating a method of monitoring a blood flow rate of a renal artery by using the blood flow rate measurement device 1.

FIG. 5 is a view illustrating denervation treatment using a neural activity stop device.

FIG. 6 is a view illustrating a medical treatment in which the neural activity stop device performs denervation on renal artery sympathetic nerves while the blood flow rate acquisition device 1 monitors a blood flow rate of a renal artery.

### DETAILED DESCRIPTION

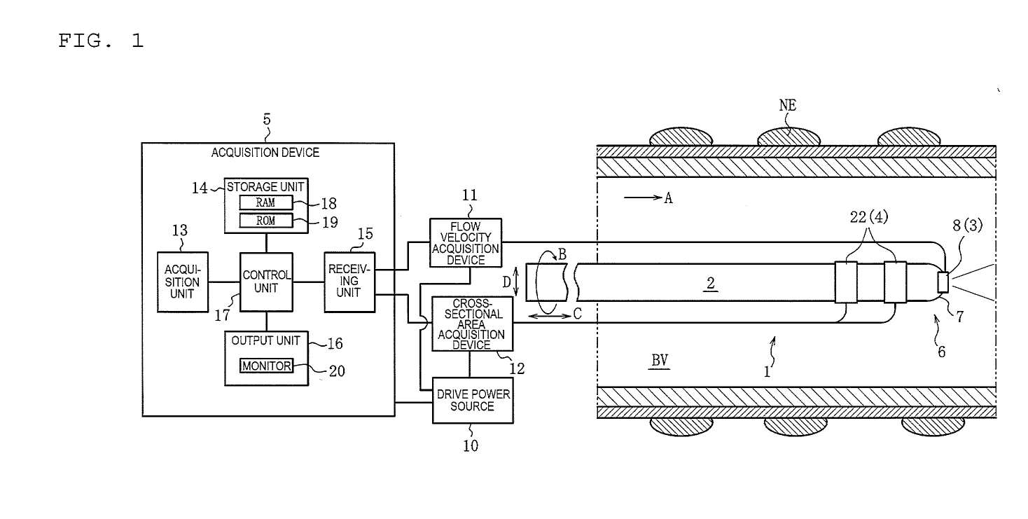

Hereinafter, embodiments of a blood flow rate measurement device will be described with reference to FIGS. 1 to 6, in which the same reference numerals are given to features common to each drawing.

FIGS. 1, 2, and 3 are views respectively illustrating blood flow rate measurement devices 1, 31, and 41 according to first, second, and third embodiments of the blood flow rate measurement device.

As illustrated in FIGS. 1, 2, and 3, each of the blood flow rate measurement devices 1, 31, and 41 includes an elongated member 2, a flow velocity measuring member 3, a cross-sectional area measuring member 4, and an acquisition device 5 which acquires a blood flow rate.

Specifically, the blood flow rate measurement devices 1, 31 and 41 respectively include an elongated member 2 that is insertable into a blood vessel BV, a flow velocity measuring member 3 that is disposed in a distal end 7 of a distal end portion 6 of the elongated member in order to measure a flow velocity of blood flow, a cross-sectional area measuring member 4 that is disposed on a side surface in the distal end portion 6 of the elongated member 2 in order to measure a cross-sectional area of a lumen of the blood vessel BV, and an acquisition device 5 that acquires the blood flow rate by using detection values detected by the flow velocity measuring member 3 and the cross-sectional area measuring member 4.

As used herein, the “flow velocity of the blood flow” refers to a velocity of the blood flow flowing in an extending (i.e., longitudinal) direction of the blood vessel BV. Additionally, as used herein, the “cross-sectional area of the lumen of the blood vessel” refers to a cross-sectional area of the lumen of the blood vessel BV in a cross section orthogonal to the extending direction of the blood vessel BV. Furthermore, as used herein, the “blood flow rate” refers to a volume of the blood flowing inside the blood vessel BV per unit time, and is derived by the product of the flow velocity of the blood flow and the cross-sectional area of the lumen of the blood vessel BV.

First, configurations common to the first to third embodiments will be described.

The elongated member 2 is a member whose distal end portion 6 is insertable into the blood vessel BV through the skin from outside of a body. For example, it is possible to use a catheter serving as a tubular member or a guidewire having no hollow portion.

FIGS. 1 to 3 illustrate the elongated member 2 using the guidewire having no hollow portion. As illustrated in FIGS. 1 to 3, the flow velocity measuring member 3 is a member which is disposed in the distal end 7 of the distal end portion 6 of the elongated member 2 and is used for measuring the blood flow velocity of the blood flow inside the blood vessel BV.

The flow velocity measuring member 3 in the blood flow rate measurement devices 1, 31, and 41 according to the first, second, and third embodiments includes a flow velocity measuring transducer 8.

The flow velocity measuring transducer 8 is attached to the distal end 7 of the elongated member 2 so as to radiate ultrasonic waves in a direction parallel to a blood flow direction A inside the blood vessel BV.

In addition, the flow velocity measuring transducer 8 receives the ultrasonic waves which are transmitted from the flow velocity measuring transducer 8 and return to the distal end 7 of the elongated member 2 by being reflected on blood components such as red blood cells contained in the blood.

As described above, the flow velocity measuring transducer 8 can transmit and receive the ultrasonic waves.

In the embodiment, the flow velocity measuring transducer 8 is driven by a drive power source 10, for example, by an A/C current having a frequency of 20 MHz to 40 MHz flowing therein.

As illustrated in FIG. 1, this drive power source 10 is a power source which supplies power to the flow velocity measuring member 3, the cross-sectional area measuring member 4 (to be described later), and the acquisition device 5 (to be described later).

Here, the flow velocity measuring transducer 8 in the first to third embodiments is disposed in the distal end 7 of the elongated member 2, and generates the ultrasonic waves in the direction parallel to the blood flow direction A inside the blood vessel BV.

According to this configuration, it is possible to reduce the number of reflection waves reflected on the components (for example, the inner wall of the blood vessel BV) other than the blood and to calculate the flow velocity of the blood flow by using the reflection waves reflected on the components in the blood, making it possible to more accurately calculate the flow velocity of the blood flow.

Furthermore, the flow velocity measuring transducer 8 is attached to the distal end 7 of the elongated member 2. Accordingly, as compared to a configuration in which the flow velocity measuring transducer 8 is attached to a side surface of the elongated member 2, there is a higher possibility that the flow velocity measuring transducer 8 is located at substantially the center portion in the cross section of the blood vessel BV. Therefore, it is possible to more stably measure the flow velocity compared to other configurations.

The flow velocity of the blood flow can be acquired by utilizing a formula of the Doppler effect, based on the frequency of the ultrasonic waves transmitted from the flow velocity measuring transducer 8, the frequency of the ultrasonic waves received by the flow velocity measuring transducer 8, and the speed of sound (i.e., 340.29 m/s).

Specifically, as illustrated in FIG. 1, a flow velocity acquisition device 11 which acquires the flow velocity of the blood flow is extracorporeally disposed, and the flow velocity acquisition device 11 calculates the flow velocity of the blood flow, based on the frequency of the ultrasonic waves transmitted from the flow velocity measuring transducer 8, the frequency of the ultrasonic waves received by the flow velocity measuring transducer 8, and the speed of sound.

In the embodiment, the frequency of the ultrasonic waves transmitted from the flow velocity measuring transducer 8 varies in response to the drive frequency of the flow velocity measuring transducer 8. Therefore, the frequency can be acquired as a value corresponding to the drive frequency.

The flow velocity acquisition device 11 acquires frequency data of the ultrasonic waves transmitted from the flow velocity measuring transducer 8, based on the drive frequency of the flow velocity measuring transducer 8.

In addition, the flow velocity acquisition device 11 is connected to the flow velocity measuring transducer 8 via a cable using a wire system or by a wireless system, and acquires the frequency data of the ultrasonic waves received by the flow velocity measuring transducer 8.

The flow velocity acquisition device 11 calculates the flow velocity of the blood flow by using the acquired data and the speed of sound.

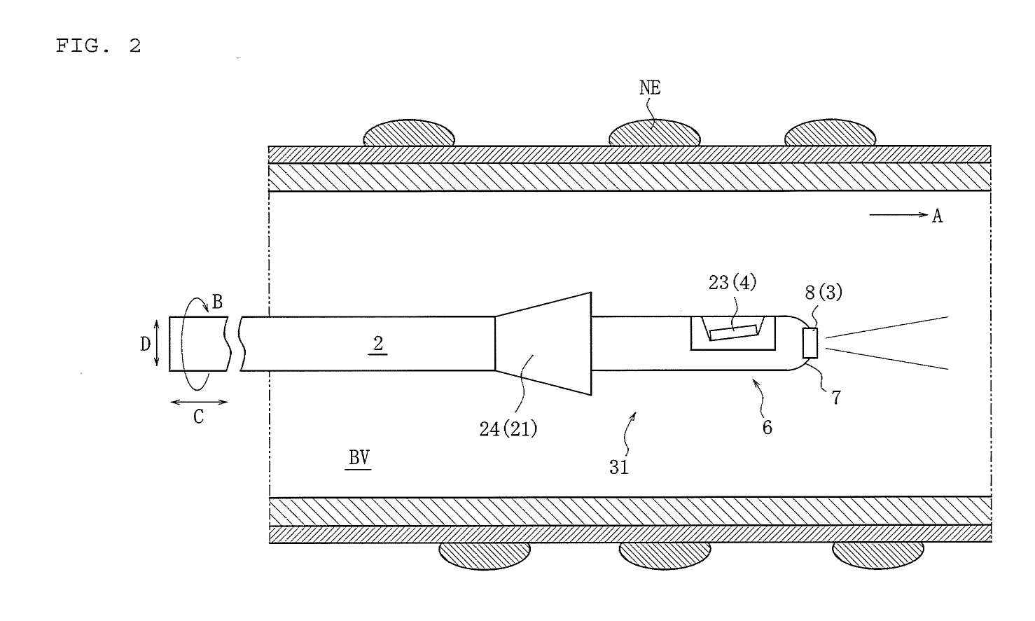

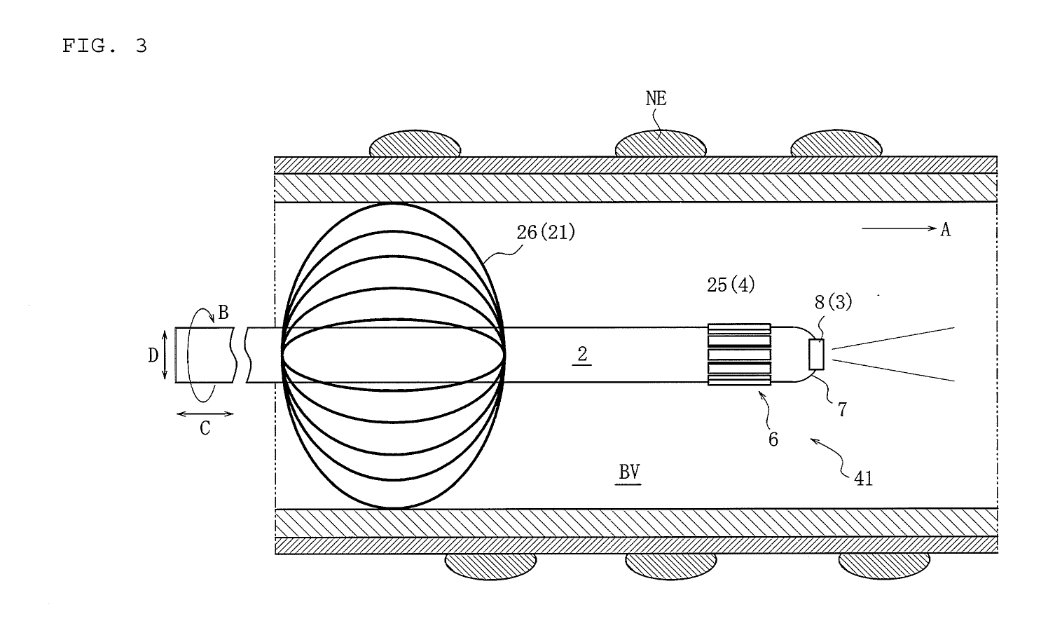

In FIG. 1, the flow velocity acquisition device 11 and the flow velocity measuring transducer 8 are illustrated as being connected to each other via the cable using the wire system. In FIGS. 2 and 3, the wire for connecting the flow velocity acquisition device 11, and the wire for connecting the flow velocity acquisition device 11 and the flow velocity measuring transducer 8 are omitted in the illustration. However, similar to the blood flow rate measurement device 1 illustrated in FIG. 1, the blood flow rate measurement devices 31 and 41 also actually have a configuration including the extracorporeally disposed flow velocity acquisition device 11 and the wire for connecting the flow velocity acquisition device 11 and the flow velocity measuring transducer 8.

As illustrated in FIGS. 1 to 3, the cross-sectional area measuring member 4 is a member which is disposed on the side surface in the distal end portion 6 of the elongated member 2 and is used for measuring the cross-sectional area of the lumen of the blood vessel BV.

Specifically, the cross-sectional area measuring member 4 acquires information used for acquiring the cross-sectional area of the lumen of the blood vessel BV.

As illustrated in FIG. 1, the information acquired by a cross-sectional area measuring member 4 is transmitted to the cross-sectional area acquisition device 12 which is extracorporeally disposed and connected to the cross-sectional area measuring member 4 via a cable using a wire system or by a wireless system, and the cross-sectional area of the lumen of the blood vessel BV is acquired by the cross-sectional area acquisition device 12.

A specific configuration of the cross-sectional area measuring member 4 will be described in detail in each description of the first to third embodiments.

In FIGS. 2 and 3, the cross-sectional area acquisition device 12, and the wire for connecting the cross-sectional area acquisition device 12 and the cross-sectional area measuring member 4 are omitted in the illustration. However, similar to the blood flow rate measurement device 1 illustrated in FIG. 1, the blood flow rate measurement devices 31 and 41 also actually have a configuration including the extracorporeally disposed cross-sectional area acquisition device 12 and the wire for connecting the cross-sectional area acquisition device 12 and the cross-sectional area measuring member 4.

As illustrated in FIG. 1, the acquisition device 5 acquires the blood flow rate by using detection values detected by the flow velocity measuring member 3 and the cross-sectional area measuring member 4.

Specifically, the acquisition device 5 includes an acquisition unit 13, a storage unit 14, a receiving unit 15, an output unit 16, and a control unit 17.

In FIGS. 2 and 3, the acquisition device 5 is omitted in the illustration. However, similar to the blood flow rate measurement device 1 illustrated in FIG. 1, the blood flow rate measurement devices 31 and 41 also actually have a configuration including the acquisition device 5.

The acquisition unit 13 calculates and acquires the blood flow rate inside the blood vessel BV, based on the flow velocity of the blood flow calculated by the flow velocity acquisition device 11 using the detection value detected by the flow velocity measuring member 3, and the cross-sectional area of the lumen of the blood vessel BV which is acquired by the cross-sectional area acquisition device 12 using the information acquired by the cross-sectional area measuring member 4. For example, the blood flow rate inside the blood vessel BV can be calculated as the product of the flow velocity of the blood flow and the cross-sectional area of the lumen of the blood vessel BV

In this manner, it is possible to immediately calculate the blood flow rate inside the blood vessel BV, based on the detection values detected by the flow velocity measuring member 3 and the cross-sectional area measuring member 4.

As illustrated in FIG. 1, the storage unit 14 includes a RAM 18 (“RAM” is an abbreviation of Random Access Memory) and a ROM 19 (“ROM” is an abbreviation of Read Only Memory).

The RAM 18 is means for temporarily storing frequency information of the ultrasonic waves which is acquired by the flow velocity measuring member 3, the flow velocity of the blood flow which is calculated by the flow velocity acquisition device 11, information acquired by the cross-sectional area measuring member 4, and the cross-sectional area of the lumen of the blood vessel BV which is acquired by the cross-sectional area acquisition device 12.

In addition, the ROM 19 stores an overall control program for the acquisition device 5, a control program for the flow velocity acquisition device 11, a control program for the cross-sectional area acquisition device 12, and an association table in which the frequency of the NC current supplied to the flow velocity measuring transducer 8, the drive frequency of the flow velocity measuring transducer 8, and the frequency of the ultrasonic waves which are transmitted from the flow velocity measuring transducer 8 are associated with one another in advance.

The receiving unit 15 receives data of the flow velocity of the blood flow which is calculated by the flow velocity acquisition device 11 and data of the cross-sectional area of the lumen of the blood vessel BV which is acquired by the cross-sectional area acquisition device 12 respectively from the flow velocity acquisition device 11 and the cross-sectional area acquisition device 12.

The output unit 16 includes a monitor 20 which outputs the blood flow rate of the blood vessel BV which is acquired by the acquisition unit 13.

A health care worker such as a surgeon can monitor the blood flow rate of a patient through the monitor 20.

The control unit 17 is connected to the flow velocity acquisition device 11, the cross-sectional area acquisition device 12, the acquisition unit 13, the storage unit 14, the receiving unit 15, and the output unit 16, respectively, and controls these devices and units.

The first to third embodiments have a configuration in which the acquisition device 5, the flow velocity acquisition device 11, and the cross-sectional area acquisition device 12 are separate from one another, the acquisition device 5 and the flow velocity acquisition device 11 are connected to each other via a cable using a wire system, and the acquisition device 5 and the cross-sectional area acquisition device 12 are connected to each other via the cable using the wire system. However, in further embodiments, the acquisition device 5 and the flow velocity acquisition device 11 are connected to each other by a wireless system, and the acquisition device 5 and the cross-sectional area acquisition device 12 are connected to each other by the wireless system.

In addition, the acquisition device 5 may be configured to include a flow velocity acquisition unit which has a function similar to that of the flow velocity acquisition device 11 and acquires the flow velocity of the blood flow inside the blood vessel BV, and a cross-sectional area acquisition unit which has a function similar to that of the cross-sectional area acquisition device 12 and acquires the cross-sectional area of the lumen of the blood vessel BV.

Furthermore, instead of the flow velocity acquisition device 11 and the cross-sectional area acquisition device 12, the acquisition unit 13 in the acquisition device 5 may be configured to include a function for calculating the flow velocity of the blood flow using the detection value detected by the flow velocity measuring member 3 and a function for acquiring the cross-sectional area of the lumen of the blood vessel BV using the detection value detected by the cross-sectional area measuring member 4.

In addition, the first to third embodiments have a configuration in which the acquisition device 5 itself includes the monitor 20. However, in further embodiments, the acquisition device 5 includes a transmission unit, the control unit 17 transmits the data of the blood flow rate which is acquired by the acquisition unit 13 to an external device having a monitor through the transmission unit, and the monitor in the external device monitors the blood flow rate.

Hitherto, configurations common to the blood flow rate measurement devices 1, 31, and 41 according to the first to third embodiments have been described.

Hereinafter, in each embodiment, configurations different from those of the other embodiments will be mainly described. Since the configurations common to the other embodiments have been described above, description thereof will be omitted herein.

Specifically, the cross-sectional area measuring member 4 in each embodiment and a stabilizing member 21 which stabilizes a position of the distal end portion 6 of the elongated member 2 inside the blood vessel BV will be described.

As illustrated in FIG. 1, the cross-sectional area measuring member 4 in the blood flow rate measurement device 1 according to the first embodiment is a pair of annular electrodes 22 surrounding the side surface of the elongated member 2 in a circumferential direction B. The pair of annular electrodes 22 are arranged to be apart from each other in an extending (i.e., longitudinal) direction C of the elongated member 2.

The pair of electrodes 22 are electrodes for measuring conductance between electrodes.

Specifically, the A/C current having a predetermined frequency (for example, several tens of KHz) is caused to flow in the pair of electrodes 22 by the drive power source 10. The conductance between the pair of electrodes 22 when the A/C current flows is measured.

The conductance is the reciprocal of electrical resistance, and is an indicator indicating a state where the current is unlikely to flow therein (or, likely to flow therein).

If the cross-sectional area of the lumen of the blood vessel BV is large, the current is likely to flow therein, and if the cross-sectional area of the lumen of the blood vessel BV is small, the current is unlikely to flow therein.

Therefore, if the conductance and the cross-sectional area of the lumen of the blood vessel BV are associated with each other in advance, it is possible to acquire the cross-sectional area of the lumen of the blood vessel BV by measuring the conductance between the pair of electrodes 22.

In the present embodiment, the ROM 19 of the acquisition device 5 stores the association table in which a value of the conductance between the pair of electrodes 22 and the cross-sectional area of the blood vessel BV are associated with each other. The cross-sectional area acquisition device 12 acquires the cross-sectional area of the lumen of the blood vessel BV by using the value of the conductance measured between the pair of electrodes 22.

The pair of electrodes 22 in the present embodiment are arranged to be apart from each other by a distance of 1 mm to 10 mm in the extending direction C.

In addition, as a material of the electrodes 22, it is possible to use a noble metal-based material such as platinum, platinum, iridium, and the like.

Each of the electrodes 22 in the present embodiment has a ring shape covering the peripheral surface of the distal end portion 6 of the elongated member 2. This shape enables the electrodes 22 to acquire the conductance corresponding to the entire cross section of the lumen of the blood vessel BV. Therefore, for example, as compared to a case of using a C-shaped electrode having a partial gap, it is possible to more accurately derive the cross-sectional area.

In addition, the cross-sectional area measuring member 4 using the pair of electrodes 22 in the present embodiment is unlikely to receive influence caused by a thrombus or the like inside the blood vessel BV, as compared to the cross-sectional area measuring member 4 which acquires the cross-sectional area of the lumen of the blood vessel BV by utilizing an IVUS (abbreviation of Intravascular Ultrasound, which means “ultrasonic waves inside the blood vessel”) in the second and third embodiments (to be described later). Therefore, it is possible to more accurately acquire the cross-sectional area of the lumen of the blood vessel BV.

As illustrated in FIG. 2, the cross-sectional area measuring member 4 in the blood flow rate measurement device 31 according to the second embodiment includes a single unit of a cross-sectional area measuring transducer 23 which generates the ultrasonic waves in a direction D substantially orthogonal to the extending direction C of the elongated member 2. Rotation of the elongated member 2 by the surgeon enables the cross-sectional area measuring transducer 23 to generate the ultrasonic waves toward the entire region in the circumferential direction of the inner wall of the blood vessel BV.

Specifically, the cross-sectional area measuring transducer 23 is disposed on the side surface in the distal end portion of the elongated member 2, and is driven by a predetermined drive frequency transmitted from the drive power source 10. In this manner, the cross-sectional area measuring transducer 23 is vibrated in the direction D orthogonal to the extending direction C of the elongated member 2, or in a direction tilted to the proximal side by a predetermined angle from the direction D orthogonal to the extending direction C, thereby transmitting the ultrasonic waves toward the inner wall of the blood vessel BV.

In addition, the cross-sectional area measuring transducer 23 receives the ultrasonic waves (reflection waves) which are transmitted from the cross-sectional area measuring transducer 23 and are reflected on the inner wall of the blood vessel.

A time period (i.e., a time period for reciprocating) after the ultrasonic waves are transmitted from the cross-sectional area measuring transducer 23 until the ultrasonic waves return to the position of the cross-sectional area measuring transducer 23 by being reflected on the inner wall of the blood vessel BV varies according to a distance between the cross-sectional area measuring transducer 23 and the inner wall of the blood vessel BV in the direction D orthogonal to the extending direction C of the elongated member 2. The cross-sectional area measuring transducer 23 generates a pixel signal according to the time period for reciprocating.

The cross-sectional area measuring transducer 23 in the present embodiment rotates with the elongated member 2 in the circumferential direction B, thereby generating the pixel signal according to the time period for reciprocating at each position in the circumferential direction B.

The cross-sectional area measuring transducer 23 is connected to the cross-sectional area acquisition device 12 by a wire (refer to FIG. 1).

The cross-sectional area acquisition device 12 receives the pixel signal generated by the cross-sectional area measuring transducer 23.

The cross-sectional area acquisition device 12 acquires a cross-sectional image of the lumen of the blood vessel BV, based on the pixel signal at each position in the circumferential direction B of the elongated member 2.

In addition, the cross-sectional area acquisition device 12 calculates the cross-sectional area of the lumen of the blood vessel BV by using image processing such as edge detection from the information of the cross-sectional image, and acquires the cross-sectional area of the lumen of the blood vessel BV.

In addition, as illustrated in FIG. 2, the blood flow rate measurement device 31 according to the second embodiment includes the stabilizing member 21 which stabilizes a position of the distal end portion 6 of the elongated member 2 inside the blood vessel BV.

The stabilizing member 21 in the present embodiment is a hollow truncated cone-shaped cover member 24 which stabilizes the position of the distal end portion 6 of the elongated member 2 inside the blood vessel BV.

Specifically, the cover member 24 serving as the stabilizing member 21 has an outer shape having a substantially truncated cone shape in which a hollow portion penetrating a top surface and a bottom surface is divided.

The elongated member 2 extends to penetrate the hollow portion, and the cover member 24 is attached to the side surface of the elongated member 2 so that an outer diameter of the cross section orthogonal to the extending direction C gradually increases toward the distal end 7 side in the extending direction C of the elongated member 2.

The cover member 24 in the present embodiment is configured so that a portion of an inner surface of the cover member 24 comes into contact with an outer surface of the elongated member 2. However, without being limited to this configuration, the entire region of the inner surface of the cover member 24 may be configured to come into contact with the outer surface of the elongated member 2.

The cover member 24 is located inside the blood vessel BV in a state of not being in contact with the inner wall of the blood vessel BV.

The cover member 24 can stay in the vicinity of the center portion in the cross section of the blood vessel BV in such a manner that the action of the blood flow causes the blood inside the blood vessel BV to collide with the outer peripheral surface of the cover member 24 with a substantially uniform force in the entire region in the circumferential direction B of the elongated member 2.

Therefore, even when the elongated member 2 is rotated, the flow velocity measuring transducer 8 and the cross-sectional area measuring transducer 23 can be stably located in the vicinity of the center portion in the cross section of the blood vessel BV by the cover member 24. Accordingly, it is possible to prevent the position inside the cross section of the lumen of the blood vessel BV from causing variations in an acquisition value of the flow velocity of the blood flow and an acquisition value of the cross-sectional area of the lumen of the blood vessel BV. As a result, it is possible to improve accuracy in a calculation value of the blood flow rate inside the blood vessel BV which is calculated based on these acquisition values.

As illustrated in FIG. 3, the cross-sectional area measuring member 4 in the blood flow rate measurement device 41 according to the third embodiment includes a cross-sectional area measuring transducer 25 which generates the ultrasonic waves in the direction D substantially orthogonal to the extending direction C of the elongated member 2. The cross-sectional area measuring transducer 25 is disposed at multiple locations in the circumferential direction B of the side surface of the elongated member 2.

Similar to the cross-sectional area measuring transducer 23 in the above-described second embodiment, each cross-sectional area measuring transducer 25 is vibrated in the direction D substantially orthogonal to the extending direction C of the elongated member 2, transmits the ultrasonic waves toward the inner wall of the blood vessel BV, receives the ultrasonic waves (reflection waves) reflected on the inner wall of the blood vessel, and generates the pixel signal according to the distance between each cross-sectional area measuring transducer 25 and the inner wall of the blood vessel BV in the direction D substantially orthogonal to the extending direction C of the elongated member 2.

Thereafter, the cross-sectional area acquisition device 12 receives the pixel signal generated by each cross-sectional area measuring transducer 25, and acquires the cross-sectional image of the lumen of the blood vessel BV.

In addition, similar to the above-described second embodiment, the cross-sectional area acquisition device 12 calculates the cross-sectional area of the lumen of the blood vessel BV by using the image processing such as the edge detection from the information of the cross-sectional image, and acquires the cross-sectional area of the lumen of the blood vessel.

In the present embodiment disclosed herein, the multiple cross-sectional area measuring transducers 25 are disposed in the circumferential direction B of the elongated member 2. Therefore, it is possible to acquire the cross-sectional image of the blood vessel without rotating the elongated member 2.

For this reason, a surgeon does not need to rotate the elongated member 2 in order to acquire the cross-sectional image of the lumen of the blood vessel BV. Therefore, it is possible to suppress variations in the cross-sectional image of the blood vessel BV which may occur depending on the surgeon's manipulation skill.

However, in the present embodiment, the control unit of the cross-sectional area acquisition device 12 controls the timing for vibrating each cross-sectional area measuring transducer 25.

If the ultrasonic waves are simultaneously transmitted from the multiple cross-sectional area measuring transducers 25, it is not possible to determine which cross-sectional area measuring transducer 25 transmits the ultrasonic waves. Consequently, it may not be possible to accurately measure the distance between the cross-sectional area measuring transducers 25 and the inner wall of the blood vessel BV in the direction D substantially orthogonal to the extending direction C of the elongated member 2.

For this reason, in the present embodiment, the control unit of the cross-sectional area acquisition device 12 controls the multiple cross-sectional area measuring transducers 25 so as to be sequentially driven along the circumferential direction B of the elongated member 2.

The control of the timing for vibrating each cross-sectional area measuring transducer 25 is not limited to the above-described control for sequentially driving each cross-sectional area measuring transducer 25 in the circumferential direction B. The control can be appropriately changed depending on a configuration or performance of the cross-sectional area measuring transducer 25 to be used.

For example, the multiple adjacent cross-sectional area measuring transducers 25 can be simultaneously vibrated, and one cross-sectional area measuring transducer 25 out of these multiple cross-sectional area measuring transducers 25 caused to receive the reflection waves. In this manner, it is possible to strengthen a receiving signal.

In addition, as illustrated in FIG. 3, the blood flow rate measurement device 41 according to the third embodiment includes the stabilizing member 21 which stabilizes the position of the distal end portion 6 of the elongated member 2 inside the blood vessel BV.

The stabilizing member 21 in the present embodiment is an expansion member 26 which expands at a predetermined position inside the blood vessel and comes into contact with the inner wall of the blood vessel BV.

As illustrated in FIG. 3, the expansion member 26 is attached to the side surface of the distal end portion 6 of the elongated member 2 so as to surround the outer peripheral surface of the elongated member 2.

The expansion member 26 substantially uniformly expands at a predetermined position inside the blood vessel in the entire region in the circumferential direction B, and is changed to have a substantially spherical outer shape in the direction D orthogonal to the extending direction C of the elongated member 2 as illustrated in FIG. 3.

The expansion member 26 comes into contact with the inner wall of the blood vessel BV and expands to a position for pressing the inner wall of the blood vessel BV.

In this state, the position of the expansion member 26 inside the blood vessel BV is fixed by a frictional force generated between the expansion member 26 and the inner wall of the blood vessel BV.

The expansion member 26 expands as described above, and the position inside the blood vessel BV is fixed. In this manner, the position of the distal end portion 6 of the elongated member 2 to which the expansion member 26 is attached is also stabilized in the vicinity of the center portion of the cross section of the blood vessel BV.

The expansion member 26 in the present embodiment is configured to have a wire member. Accordingly, even in a state where the expansion member 26 expands, the blood flow is ensured through a gap of the expansion member 26.

In addition, the expansion member 26 in the present embodiment employs a self-expansion type of the expansion member 26. That is, the expansion member 26 in the present embodiment is interposed between the outer surface of the elongated member 2 and the inner surface of an outer tubular member surrounding the elongated member 2 in the circumferential direction B. In this manner, the expansion member 26 is inserted into a predetermined position inside the blood vessel BV in a state where each wire member is elastically deformed so as to extend to be substantially parallel to the extending direction C of the elongated member 2.

Then, at this predetermined position, the outer tubular member is pulled out to the proximal side with respect to the elongated member 2, or the elongated member 2 to which the expansion member 26 is attached is pushed to the distal side with respect to the outer tubular member. In this manner, the expansion member 26 is released from the outer tubular member, and is caused to expand by a restoring force.

Here, the expansion member 26 in the present embodiment employs one which is changed to have a substantially spherical outer shape when expanded. However, without being limited to the above-described expansion member 26, for example, it is also possible to use an expansion member configured to have multiple linear members which are attached to the side surface of the elongated member 2 and radially extend in the direction D orthogonal to the extending direction C of the elongated member 2 when expanded, or in a direction tilted to the distal side by a predetermined angle from the direction D orthogonal to the extending direction C.

A material of the wire member includes a synthetic resin, metal, and the like. As a synthetic resin, for example, polyolefin, polyester, and a fluorine resin can be used. One type of these may be used alone, or two or more types may be used in combination.

The polyolefin is not particularly limited, and can be appropriately selected depending on a purpose of use. For example, the polyolefin can include polyethylene, polypropylene, and the like.

In addition, the polyester is not particularly limited, and can be appropriately selected depending on a purpose of use. For example, the polyester can include polyethylene terephthalate, polybutylene terephthalate, and the like.

Similarly, the fluorine resin is not particularly limited, and can be appropriately selected depending on a purpose of use. For example, the fluorine resin can include polytetrafluoroethylene (PTFE), a copolymer of tetrafluoroethylene and ethylene (ETFE), and the like.

It is preferable to use a resin having predetermined hardness and elasticity or a resin having biocompatibility as the other properties.

In addition, as the metal, for example, stainless steel, tantalum, a nickel titanium alloy, elastic metal, and the like can be used. One type of these may be used alone, or two or more types may be used in combination.

It is preferable to use the elastic metal among these, and it is more preferable to use a super-elastic alloy.

The super-elastic alloy is generally called a shape memory alloy, and demonstrates elasticity at least at a biological temperature (approximately 37° C.).

The super-elastic alloy is not particularly limited. However, it is preferable to use a titanium-nickel alloy containing nickel of 49 atomic % to 53 atomic %.

As described above, the blood flow rate measurement device can be realized by various and specific configurations, and is not limited to the configurations described in the above embodiments.

For example, the blood flow rate measurement device 1 according to the first embodiment illustrated in FIG. 1 has a configuration excluding the stabilizing member 21, but can also have a configuration including the cover member 24 in the blood flow rate measurement device 31 according to the second embodiment or the expansion member 26 in the blood flow rate measurement device 41 according to the third embodiment. Accordingly, it is possible to provide a blood flow rate measurement device which combines features of the configurations described in each embodiment.

Next, a monitoring method of the blood flow rate of the renal artery RA serving as the blood vessel BV using the blood flow rate measurement device 1 according to the first embodiment will be described.

Specifically, a case will be described in which in medical treatment for inactivating a renal artery sympathetic nerve NE for a resistant hypertension patient (the nerve inactivating is hereinafter referred to as “denervation”), the blood flow rate of the renal artery RA is monitored in order to determine whether or not this denervation treatment is completed.

With regard to the denervation treatment for the renal artery sympathetic nerve NE, there is a problem in that the denervation treatment has no method for determining whether or not the denervation is reliably completed during the medical treatment or immediately after the medical treatment, and that it is difficult to determine whether or not a patient who feels no treatment effect even after the medical treatment needs additional medical treatment.

The monitoring method described herein enables a health care worker such as a surgeon to easily determine whether or not the denervation is reliably performed during the medical treatment or immediately after the medical treatment.

First, a relationship between the renal artery sympathetic nerve NE and the blood flow rate inside the renal artery RA will be described.

Many arterioles for filtering the blood are present in a kidney.

A diameter of the arterioles is governed by the renal artery sympathetic nerve NE around the renal artery RA.

Specifically, if a neural activity of the renal artery sympathetic nerve NE becomes active, the arterioles become hardened, and the diameter decreases (the blood vessel is less likely to be deformed due to the pressure of the blood, and the lumen of the arterioles is in a narrowed state).

Then, if the diameter of the arterioles decreases, the blood flow rate flowing in the renal artery RA also decreases.

On the other hand, when the neural activity of the renal artery sympathetic nerve NE is in an inactive state, that is, when the denervation is performed, the arterioles inside the kidney become softened. Thus, the blood pressure can smoothly widen the diameter.

Therefore, when the denervation of the renal artery sympathetic nerve NE is completed, the blood pressure causes the arterioles to increase the diameter. Accordingly, the blood flow rate of the renal artery RA also increases.

Therefore, a health care worker such as a surgeon monitors the blood flow rate of the renal artery RA, and thus can determine whether or not the denervation treatment of the renal artery sympathetic nerve NE is completed.

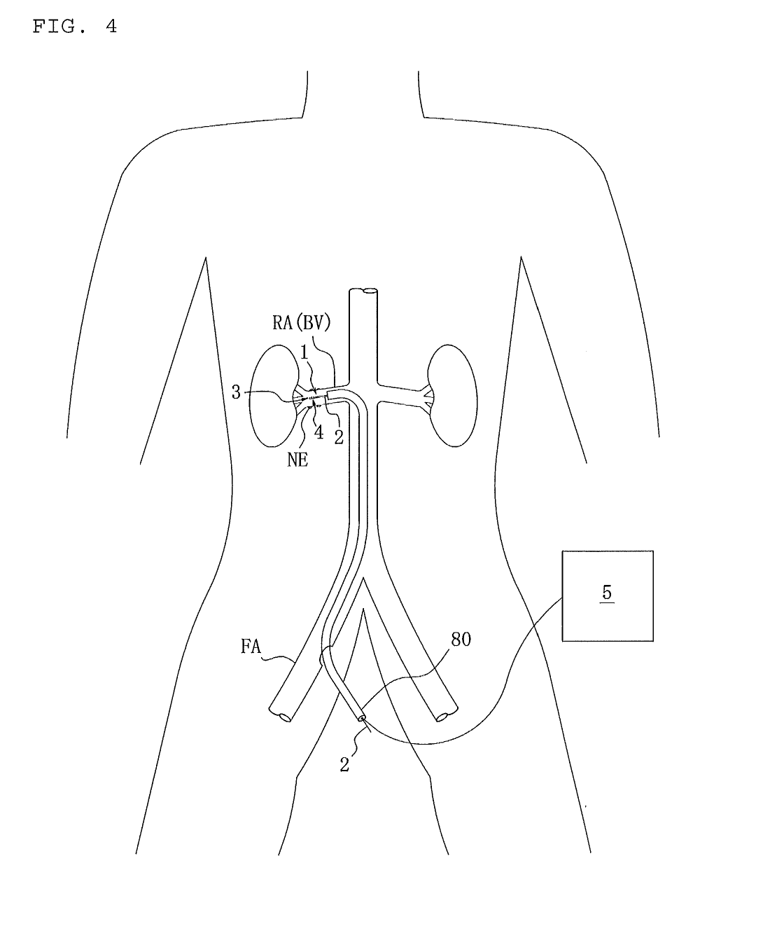

FIG. 4 is a view illustrating a method of monitoring the blood flow rate of the renal artery RA using the blood flow rate measurement device 1 according to the first embodiment.

This method includes a process of guiding the flow velocity measuring member 3 for measuring the blood flow velocity (flow velocity of the blood flow) inside the renal artery RA serving as the blood vessel BV and the cross-sectional area measuring member 4 for measuring the cross-sectional area of the renal artery RA to a predetermined position inside the renal artery RA, and a process of monitoring the blood flow rate acquired by using detection values detected by the flow velocity measuring member 3 and the cross-sectional area measuring member 4.

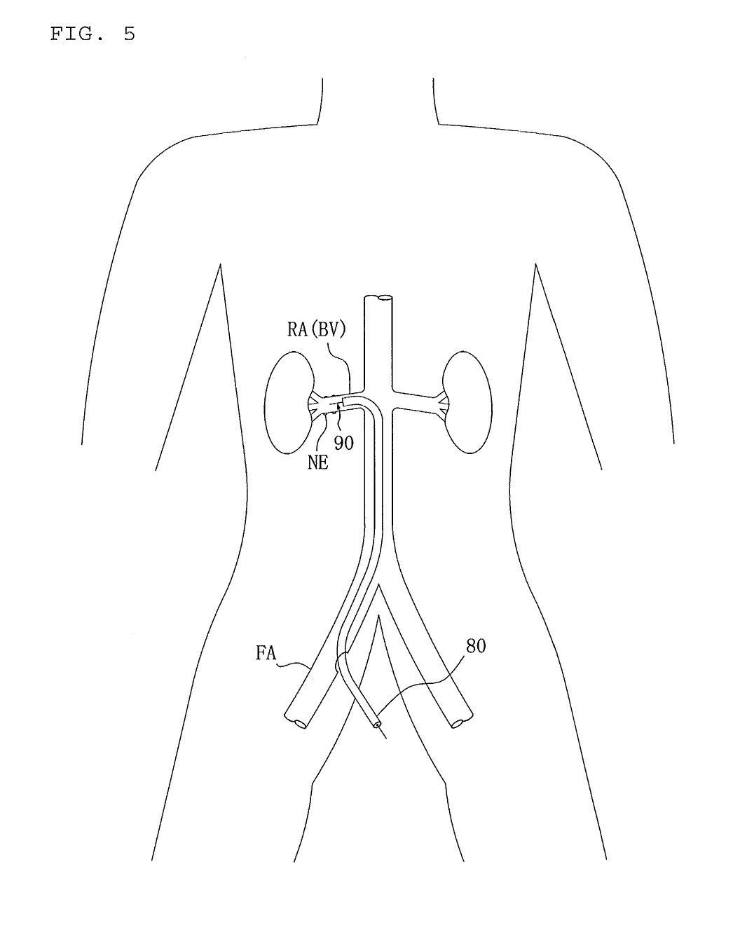

Specifically, before the denervation treatment (refer to FIG. 5) of the renal artery sympathetic nerve NE (to be described later) is performed, a surgeon inserts the guidewire serving as the elongated member 2 in which the flow velocity measuring member 3 and the cross-sectional area measuring member 4 are disposed in the distal end portion 6 into the femoral artery FA of a patient, and causes the distal end portion 6 of the elongated member 2 to reach the predetermined position inside the renal artery RA.

Then, the surgeon acquires the flow velocity of the blood flow at the predetermined position inside the renal artery RA by using the flow velocity measuring transducer 8 in the flow velocity measuring member 3.

In addition, simultaneously with the process of acquiring the flow velocity of the blood flow, or at any time before or after the process, the surgeon acquires the cross-sectional area of the lumen at the predetermined position inside the renal artery RA by using the pair of electrodes 22 in the cross-sectional area measuring member 4.

The specific acquisition method for the flow velocity of the blood flow and the cross-sectional area of the lumen of the renal artery RA has been described in the first to third embodiments. Therefore, description thereof will be omitted herein.

Then, the acquisition device 5 acquires the blood flow rate at the predetermined position of the renal artery RA.

The acquisition method for acquiring the blood flow rate using the acquisition device 5 has also been described in the first to third embodiments. Therefore, description thereof will be omitted herein.

As described above, a health care worker such as a surgeon performs the above-described monitoring before and after the denervation treatment (refer to FIG. 5: to be described later), and thus can easily determine whether or not the denervation treatment of the renal artery sympathetic nerve NE is reliably completed.

In particular, according to the blood flow rate measurement device 1, it is possible to immediately acquire the blood flow rate. Therefore, it is possible to realize real-time monitoring of the blood flow rate.

The method for monitoring the blood flow rate has been described herein by using the blood flow rate measurement device 1 according to the first embodiment. However, instead of the blood flow rate measurement device 1, of course, it is also possible to use the blood flow rate measurement device 31 according to the second embodiment or the blood flow rate measurement device 41 according to the third embodiment.

Next, treatment for performing the denervation of the renal artery sympathetic nerve NE using a neural activity stop device 90 will be described.

FIG. 5 is a view illustrating the denervation treatment using the neural activity stop device 90, which is performed after the blood flow rate measurement device 1 monitors (refer to FIG. 4) the blood flow rate of the renal artery RA.

The denervation treatment illustrated in FIG. 5 includes a process of guiding the neural activity stop device 90 which stops the neural activity of the renal artery sympathetic nerve NE located in the vicinity of the outer wall of the renal artery RA to a predetermined position inside the renal artery RA, and a process of performing an operation for causing the neural activity stop device 90 to stop the neural activity.

For example, the neural activity stop device 90 includes a cauterizing device which cauterizes the renal artery sympathetic nerve NE and stops the neural activity.

A surgeon inserts a distal end of the cauterizing device into a predetermined position inside the renal artery RA through a guiding catheter 80, radiates cauterizing energy (for example, ultrasonic waves) to the renal artery sympathetic nerve NE to be cauterized, and performs the denervation.

If the cauterizing device completes the denervation, the surgeon extracorporeally removes the cauterizing device through the guiding catheter 80.

Thereafter, as illustrated in FIG. 4, the surgeon re-inserts the distal end portion 6 of the blood flow rate measurement device 1, through the guiding catheter 80, into a position where the cauterizing device serving as the neural activity stop device 90 performs the denervation treatment.

Then, the surgeon monitors the blood flow rate of the renal artery RA in the same manner as before the denervation treatment, and compares the blood flow rates inside the renal artery RA which are measured before and after the denervation treatment.

As a result, if the blood flow rate inside the renal artery RA increases as compared to the blood flow rate measured before the denervation treatment, the surgeon can determine that the cauterizing device completes cauterizing for the neural activity.

In addition, when there is no significant change in the blood flow rate before and after the denervation treatment, the surgeon can determine that additional measures such as re-performing the cauterizing on the neural activity using the cauterizing device are required.

As described above, the health care worker such as the surgeon monitors the blood flow rate of the renal artery RA using the blood flow rate measurement device 1, and thus can easily determine whether or not the denervation treatment is completed.

As illustrated in FIG. 4, when the blood flow rate is monitored by using the blood flow rate measurement device 1 before the denervation treatment is performed by the neural activity stop device 90, immediately after the blood flow rate measurement device 1 is inserted into the renal artery RA, it is preferable to insert the guiding catheter 80 into the renal artery RA by following the elongated member 2 (guidewire) of the blood flow rate measurement device 1.

In this manner, after the blood flow rate inside the renal artery RA is monitored before the denervation treatment, the blood flow rate measurement device 1 can be easily and extracorporeally removed through a hollow portion of the guiding catheter 80.

Furthermore, even when the distal end of the neural activity stop device 90 illustrated in FIG. 5 is inserted into the renal artery RA after the blood flow rate measurement device 1 is extracorporeally removed, the distal end of the neural activity stop device 90 can be easily inserted through the hollow portion of the guiding catheter 80.

Referring to FIGS. 4 and 5, a case has been described in which the blood flow rate inside the renal artery RA is monitored by inserting the blood flow rate measurement device 1 which is separate from the neural activity stop device 90 into the renal artery RA before or after the denervation treatment for the renal artery sympathetic nerve NE is performed by using the neural activity stop device 90.

Herein, a method of real-time monitoring for the blood flow rate of the renal artery RA during the denervation treatment for the renal artery sympathetic nerve NE will be described.

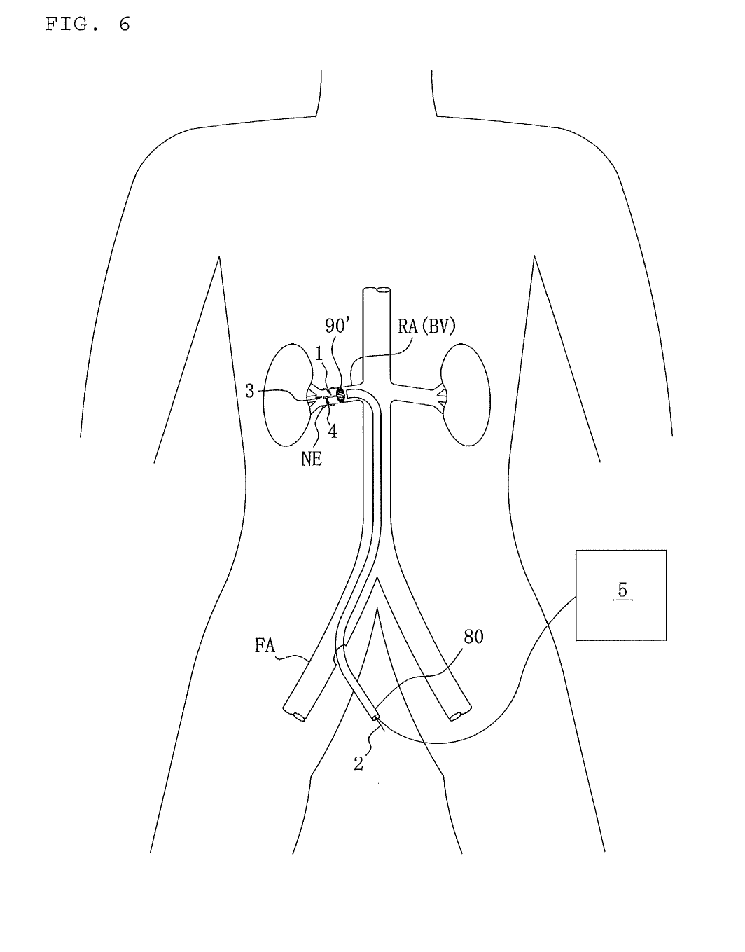

FIG. 6 is a view illustrating medical treatment in which the denervation of the renal artery sympathetic nerve NE is performed by using a neural activity stop device 90′ while the blood flow rate of the renal artery RA is monitored by using the blood flow rate measurement device 1.

The neural activity stop device 90′ is a neural activity stop device having a form different from that of the cauterizing device serving as the neural activity stop device 90 illustrated in FIG. 5. Specifically, this medical treatment includes a process of guiding the flow velocity measuring member 3 for measuring the blood flow velocity inside the renal artery RA serving as the blood vessel BV and the cross-sectional area measuring member 4 for measuring the cross-sectional area of the renal artery RA to a predetermined position inside the renal artery RA, a process of guiding the neural activity stop device 90′ which stops the neural activity of the renal artery sympathetic nerve NE located in the vicinity of the outer wall of the renal artery RA to the vicinity of the predetermined position inside the renal artery RA, a process of starting an operation for causing the neural activity stop device 90′ to stop the neural activity and monitoring the blood flow rate acquired by using detection values detected by the flow velocity measuring member 3 and the cross-sectional area measuring member 4 during the operation, and a process of determining whether the neural activity stop device 90′ completes the operation according to the monitored blood flow rate.

Hereinafter, each process will be described.

Details of the process of guiding the flow velocity measuring member 3 for measuring the blood flow velocity inside the renal artery RA serving as the blood vessel BV and the cross-sectional area measuring member 4 for measuring the cross-sectional area of the renal artery RA to a predetermined position inside the renal artery RA have been described above. Therefore, description thereof will be omitted herein (refer to FIG. 4).

Then, a surgeon guides the neural activity stop device 90′ to the vicinity of the predetermined position, inside the renal artery RA, where the flow velocity measuring member 3 and the cross-sectional area measuring member 4 are placed, internally through the guiding catheter 80, while following the elongated member 2 (guidewire) of the blood flow rate measurement device 1.

More specifically, the neural activity stop device 90′ is guided to a position between the distal end portion 6 (refer to FIG. 1) of the blood flow rate measurement device 1 and the distal end of the guiding catheter 80.

The neural activity stop device 90′ illustrated in FIG. 6 is configured so that a cauterizing electrode which cauterizes the renal artery sympathetic nerve NE is disposed on an outer surface of a self-expansion-type expansion body. Therefore, when the neural activity stop device 90′ is guided to the position between the distal end portion 6 of the blood flow rate measurement device 1 and the distal end of the guiding catheter 80, an internally contained outer tubular member is guided to the above-described position in a state where the expansion body of the neural activity stop device 90′ is contracted or folded. Then, at this position, only the outer tubular member is pulled out through the guiding catheter 80. In this manner, the expansion body of the neural activity stop device 90′ is subjected to self-expansion.

If the expansion body is expanded, the cauterizing electrode disposed on the outer surface of the expansion body comes into contact with the inner wall of the renal artery RA and further presses the inner wall. This cauterizing electrode can cauterize the renal artery sympathetic nerve NE located in the vicinity of the outer wall of the renal artery RA.

In this state, the blood flow rate measurement device 1 starts the monitoring of the blood flow rate inside the renal artery RA.

Thereafter, the surgeon starts the operation for stopping the neural activity by using the neural activity stop device 90′ while performing the monitoring using the blood flow rate measurement device 1.

The blood flow rate inside the renal artery RA is calculated by the acquisition device 5 using detection values detected by the flow velocity measuring member 3 and the cross-sectional area measuring member 4. A value of the calculated blood flow rate is monitored through the monitor 20 in the output unit 16 of the acquisition device 5 (refer to FIG. 1).

The surgeon operates the neural activity stop device 90′ while monitoring the blood flow rate inside the renal artery RA. In this manner, the surgeon determines whether or not the neural activity stop device 90′ completes the operation (that is, completes the denervation) according to the blood flow rate monitored through the monitor 20.

For example, the surgeon can cause the neural activity stop device 90′ to complete the operation when the monitored blood flow rate inside the renal artery RA reaches a predetermined amount or more.

On the other hand, when the monitored blood flow rate inside the renal artery RA is less than the predetermined amount, the surgeon causes the neural activity stop device 90′ to continue the operation.

In addition to this, for example, the surgeon may cause the neural activity stop device 90′ to complete the operation when a difference between a value of the blood flow rate monitored in the middle of the denervation treatment using the neural activity stop device 90′ and a value of the blood flow rate monitored before the denervation treatment using the neural activity stop device 90′ reaches the predetermined amount or more.

After the neural activity stop device 90′ completes the operation, the surgeon extracorporeally removes the neural activity stop device 90′ and the blood flow rate measurement device 1 from the inside of the renal artery RA through the guiding catheter 80.

In further embodiments, in addition to the blood flow rate of the renal artery RA, a blood pressure may be monitored. By monitoring the blood pressure, it is possible to derive viscoelasticity of the blood vessel, based on the blood flow rate and the blood pressure, and to more accurately diagnose a nerve block state based on the viscoelasticity.

The detailed description above describes a blood flow rate measurement device and medical treatment of a nerve. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A blood flow rate measurement device comprising:
an elongated member that is insertable into a blood vessel;
a flow velocity measuring member that is disposed in a distal end of a distal end portion of the elongated member in order to measure a flow velocity of blood flow;
a cross-sectional area measuring member that is disposed on a side surface in the distal end portion of the elongated member in order to measure a cross-sectional area of a lumen of the blood vessel; and
an acquisition device that acquires the blood flow rate by using detection values detected by the flow velocity measuring member and the cross-sectional area measuring member.

2. The blood flow rate measurement device according to claim 1,
wherein the flow velocity measuring member includes a flow velocity measuring transducer.

3. The blood flow rate measurement device according to claim 2,
wherein the flow velocity measuring transducer is attached to the distal end of the elongated member so as to radiate ultrasonic waves in a direction parallel to a blood flow direction inside the blood vessel.

4. The blood flow rate measurement device according to claim 1,
wherein the cross-sectional area measuring member includes a pair of annular electrodes surrounding the side surface of the elongated member in a circumferential direction, and
wherein the pair of annular electrodes are arranged to be apart from each other in an extending direction of the elongated member.

5. The blood flow rate measurement device according to claim 1,
wherein the cross-sectional area measuring member includes a cross-sectional area measuring transducer which generates ultrasonic waves in a direction substantially orthogonal to an extending direction of the elongated member, and
wherein the cross-sectional area measuring transducer is disposed at multiple locations in the circumferential direction of the side surface of the elongated member.

6. The blood flow rate measurement device according to claim 4, further comprising:
a stabilizing member that stabilizes a position of the distal end portion of the elongated member inside the blood vessel,
wherein the stabilizing member is an expansion member which expands at a predetermined position inside the blood vessel and comes into contact with an inner wall of the blood vessel.

7. The blood flow rate measurement device according to claim 1,
wherein the cross-sectional area measuring member includes a cross-sectional area measuring transducer which generates ultrasonic waves in a direction substantially orthogonal to an extending direction of the elongated member, and
wherein the elongated member is rotatable, thereby enabling the ultrasonic waves to be generated toward a whole region of an inner wall of the blood vessel in the circumferential direction.

8. The blood flow rate measurement device according to claim 4, further comprising:
a stabilizing member that stabilizes a position of the distal end portion of the elongated member inside the blood vessel,
wherein the stabilizing member has a substantially truncated cone shape in which a hollow portion penetrating a top surface and a bottom surface is divided, wherein the elongated member extends penetrating the hollow portion, and
wherein the stabilizing member is attached to the side surface of the elongated member so that an outer diameter of a cross section orthogonal to the extending direction gradually increases toward the distal side in the extending direction of the elongated member.

9. The blood flow rate measurement device according to claim 2,
wherein the cross-sectional area measuring member includes a pair of annular electrodes surrounding the side surface of the elongated member in a circumferential direction, and
wherein the pair of annular electrodes are arranged to be apart from each other in an extending direction of the elongated member.

10. The blood flow rate measurement device according to claim 2,
wherein the cross-sectional area measuring member includes a cross-sectional area measuring transducer which generates ultrasonic waves in a direction substantially orthogonal to an extending direction of the elongated member, and
wherein the cross-sectional area measuring transducer is disposed at multiple locations in the circumferential direction of the side surface of the elongated member.

11. The blood flow rate measurement device according to claim 2,
wherein the cross-sectional area measuring member includes a cross-sectional area measuring transducer which generates ultrasonic waves in a direction substantially orthogonal to an extending direction of the elongated member, and
wherein the elongated member is rotatable, thereby enabling the ultrasonic waves to be generated toward a whole region of an inner wall of the blood vessel in the circumferential direction.

12. The blood flow rate measurement device according to claim 5, further comprising:
a stabilizing member that stabilizes a position of the distal end portion of the elongated member inside the blood vessel,
wherein the stabilizing member is an expansion member which expands at a predetermined position inside the blood vessel and comes into contact with an inner wall of the blood vessel.

13. The blood flow rate measurement device according to claim 7, further comprising:
a stabilizing member that stabilizes a position of the distal end portion of the elongated member inside the blood vessel,
wherein the stabilizing member is an expansion member which expands at a predetermined position inside the blood vessel and comes into contact with an inner wall of the blood vessel.

14. The blood flow rate measurement device according to claim 5, further comprising:
a stabilizing member that stabilizes a position of the distal end portion of the elongated member inside the blood vessel,
wherein the stabilizing member has a substantially truncated cone shape in which a hollow portion penetrating a top surface and a bottom surface is divided, wherein the elongated member extends penetrating the hollow portion, and
wherein the stabilizing member is attached to the side surface of the elongated member so that an outer diameter of a cross section orthogonal to the extending direction gradually increases toward the distal side in the extending direction of the elongated member.

15. The blood flow rate measurement device according to claim 7, further comprising:
a stabilizing member that stabilizes a position of the distal end portion of the elongated member inside the blood vessel,
wherein the stabilizing member has a substantially truncated cone shape in which a hollow portion penetrating a top surface and a bottom surface is divided, wherein the elongated member extends penetrating the hollow portion, and
wherein the stabilizing member is attached to the side surface of the elongated member so that an outer diameter of a cross section orthogonal to the extending direction gradually increases toward the distal side in the extending direction of the elongated member.

16. A medical treatment method, comprising:
inserting an elongated member of a blood flow measurement device into a blood vessel;
measuring, with the blood flow measurement device, a flow rate of blood within the blood vessel;
performing a procedure on a portion of the blood vessel;
measuring, with the blood flow measurement device, the flow rate of blood within the blood vessel after performing the procedure; and
comparing the measured flow rate of blood within the blood vessel before performing the procedure with the measured flow rate of blood within the blood vessel after performing the procedure.

17. The medical treatment method claim 16, wherein
before performing the procedure, the elongated member of the blood flow measurement device is removed from the blood vessel; and
after performing the procedure, the elongated member of the blood flow measurement device is re-inserted into the blood vessel.

18. The medical treatment method claim 16, wherein the elongated member of the blood flow measurement device remains inserted in the blood vessel while the procedure is performed, and the method further comprises measuring the flow rate of blood within the blood vessel while the procedure is performed.

19. The medical treatment method claim 16, wherein the procedure comprises denervation of a nerve of an outer wall of the blood vessel.

20. The medical treatment method claim 19, wherein the denervation of the nerve is performed using a cauterizing electrode disposed on an outer surface of an expansion body.
